(19) United States
(12) Patent Application Publication
(10) Pub. No.: US 2016/0017399 A1
(43) Pub. Date: Jan. 21, 2016

(54) ELECTROCHEMICAL-BASED ANALYTICAL TEST STRIP WITH ENZYMATIC REAGENT LAYER CONTAINING A NAPHTHOQUINONE-BASED MEDIATOR AND FAD-GDH

(71) Applicant: LifeScan Scotland Limited, Inverness (GB)

(72) Inventors: Marco CARDOSI, Inverness (GB); Rebecca PHILLIPS, Inverness (GB); Gavin MACFIE, Inverness (GB)

(21) Appl. No.: 14/333,552

(22) Filed: Jul. 17, 2014

(51) Int. Cl. C12Q 1/00; G01N 27/327

(52) U.S. Cl. CPC C12Q 1/006; G01N 27/327

(57) ABSTRACT

An electrochemical-based analytical test strip for the determination of an analyte (such as glucose) in a bodily fluid sample includes an electrically insulating base layer, an electrically conductive layer disposed on the electrically insulating base layer and including at least one electrode, an enzymatic reagent layer disposed on the at least one electrode, a patterned spacer layer, and a top layer. Moreover, the enzymatic reagent layer includes at least one naphthoquinone-based mediator. The naphthoquinone-based mediator can, for example, be at least one of 1,2-naphthalenedione-4-(3-mercapto-1-propane sulfonic acid) and 1,2-naphthalenedione-4-(3-mercaptopropionic acid); and FAD-GDH enzyme.

### BACKGROUND OF THE INVENTION

1. Field of the Invention

The present invention relates, in general, to medical devices and, in particular, to electrochemical-based analytical test strips and enzymatic reagents for use therein.

2. Description of Related Art

The determination (e.g., detection and/or concentration measurement) of an analyte in, or a characteristic of, a fluid sample is of particular interest in the medical field. For example, it can be desirable to determine glucose, ketone bodies, cholesterol, lipoproteins, triglycerides, acetaminophen, hematocrit and/or HbA1c concentrations in a sample of a bodily fluid such as urine, blood, plasma or interstitial fluid. Such determinations can be achieved using analytical test strips, based on, for example, visual, photometric or electrochemical techniques. Conventional electrochemical-based analytical test strips are described in, for example, U.S. Pat. Nos. 5,708,247 and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified exploded perspective view of an electrochemical-based analytical test strip according to an embodiment of the present invention;

FIG. 2 is a simplified cross-sectional side view (not to scale) of a portion of the electrochemical-based analytical test strip of FIG. 1 taken along the longitudinal axis thereof;

FIG. 3 is a graph of linearity performance for enzymatic reagent according to an embodiment of the present invention;

FIG. 4 is a graph of uric acid sensitivity for an enzymatic reagent according to an embodiment of the present invention; and

FIG. 5 is a graph of linearity performance for another enzymatic reagent according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable tolerance that allows a component or collection of components to function for its intended purpose as described herein.

An electrochemical-based analytical test strip for the determination of an analyte (such as glucose) in a bodily fluid sample according to embodiments of the present invention include an electrically insulating base layer, an electrically conductive layer disposed on the electrically insulating base layer and including at least one electrode, an enzymatic reagent layer disposed on the at least one electrode, a patterned spacer layer, and a top layer. Moreover, the enzymatic reagent layer includes at least one naphthoquinone-based mediator. Such a naphthoquinone-based mediator can be, for example, 1,2-naphthalenedione-4-(3-mercapto-1-propane sulfonic acid) and 1,2-naphthalenedione-4-(3-mercaptopropionic acid), and FAD-GDH enzyme.

Electrochemical-based analytical test strips according to embodiments of the present invention are beneficial in that the combination of at least one naphthoquinone-based mediator (e.g., at least one of 1,2-naphthalenedione-4-(3-mercapto-1-propane sulfonic acid) and 1,2-naphthalenedione-4-(3-mercaptopropionic acid)), and FAD-GDH enzyme creates an enzymatic reagent layer that is biochemically efficient (for example, the mediators are 2 electron acceptors, results in an enzymatic reaction that does not proceed via a free-radical route, and has a first order rate constant is, for some naphthoquinone-based mediators, greater than that of the conventional FAD-GDH mediator potassium ferricyanide) and that is not susceptible to interference from uric acid, acetaminophen, glutathione and ascorbic acid.

Enzymatic reagents for use in electrochemical-based analytical test strips according to the present invention comprise at least one naphthoquinone-based mediator such as, for example, at least one of (i) 1,2-naphthalenedione-4-(3-mercapto-1-propoane sulfonic acid) and (ii) 1,2-naphthalenedione-4-(3-mercaptopropionic acid); and flavin adenine dinucleotide dependant glucose dehydrogenase enzyme, herein abbreviated to FAD-GDH enzyme. Such enzymatic reagents are beneficial in that they creates an enzymatic reagent layer that is biochemically efficient (i.e., the mediators are 2 electron acceptors and the enzymatic reaction does not proceed via a free-radical route) and not susceptible to interference from uric acid, acetaminophen, glutathione and ascorbic acid. Moreover, the enzymatic reagents can be applied during manufacturing of electrochemical-based analytical test strip using conventional techniques such as ink jet printing and screen printing.

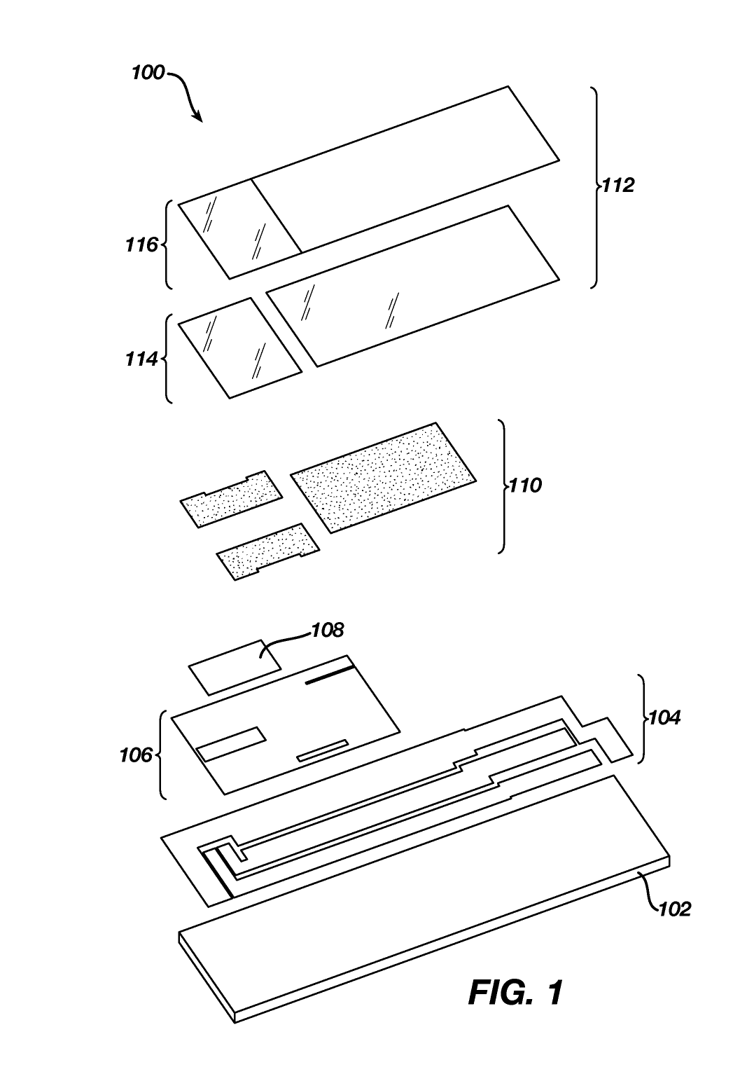

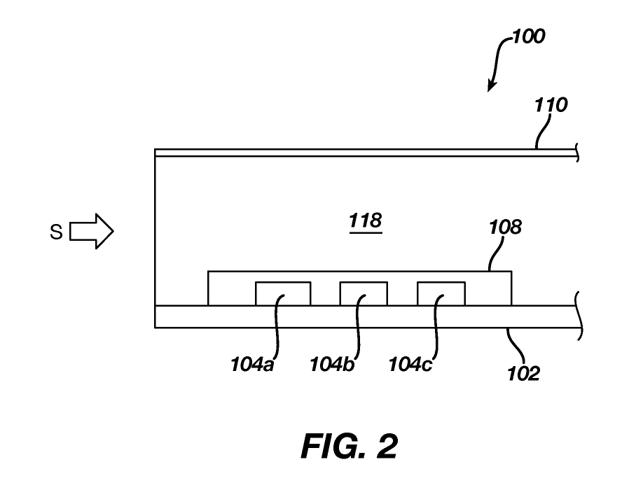

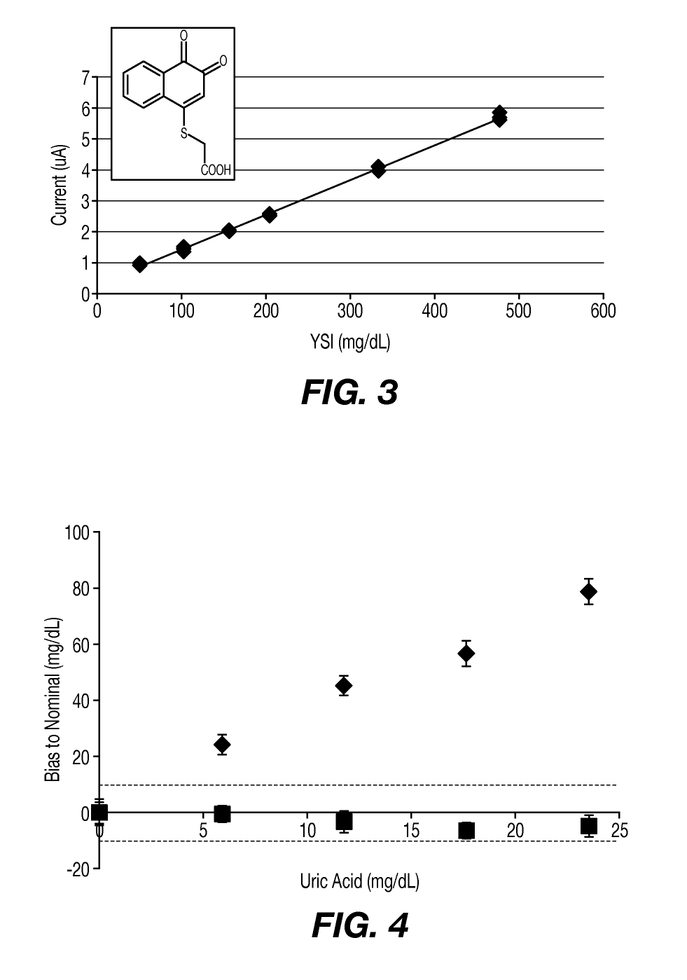

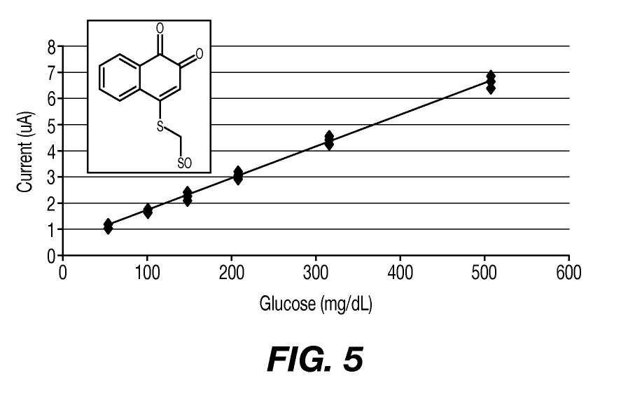

FIG. 1 is a simplified exploded perspective view of an electrochemical-based analytical test strip 100 according to an embodiment of the present invention. FIG. 2 is a simplified cross-sectional side view (not to scale) of a portion of electrochemical-based analytical test strip 100 taken along the longitudinal axis in the perspective of FIG. 1. FIG. 3 is a graph of linearity performance for enzymatic reagent according to an embodiment of the present invention. FIG. 4 is a graph of uric acid sensitivity for an enzymatic reagent according to an embodiment of the present invention. FIG. 5 is a graph of linearity performance for an enzymatic reagent according to another embodiment of the present invention.

Referring to FIGS. 1 through 5, electrochemical-based analytical test strip 100 for the determination of an analyte (such as glucose) in a bodily fluid sample (for example, a whole blood sample) includes an electrically-insulating base layer 102, a patterned electrically conductive layer 104, a patterned insulation layer 106, an enzymatic reagent layer 108, a patterned spacer layer 110, and a top layer 112 consisting of a hydrophilic sub-layer 114 and a top tape 116.

In the embodiment of FIGS. 1 and 2, at least the patterned spacer layer and top layer define a sample-receiving chamber 118 within electrochemical-based analytical test strip 100 (see FIG. 2 in particular).

Electrically-insulating base layer 102 can be any suitable electrically-insulating base layer known to one skilled in the art including, for example, a nylon base layer, a polycarbonate base layer, a polyimide base layer, a polyvinyl chloride base layer, a polyethylene base layer, a polypropylene base layer, a glycolated polyester (PETG) base layer, or a polyester base layer. The electrically-insulating base layer can have any suitable dimensions including, for example, a width dimension of about 5 mm, a length dimension of about 27 mm and a thickness dimension of about 0.5 mm.

Electrically-insulating base layer 102 provides structure to electrochemical-based analytical test strip 100 for ease of handling and also serves as a base for the application (e.g., printing or deposition) of subsequent layers (e.g., a patterned electrically conductor layer and an enzymatic reagent formed by ink jet printing or screen printing of an enzymatic reagent according to the present invention and described herein).

Patterned electrically conductive layer 104 is disposed on the electrically-insulating base layer 102 and includes a first electrode 104a, a second electrode 104b and a third electrode 104c. First electrode 104a, second electrode 104b and third electrode 104c can be, for example, configured as a counter/reference electrode, a first working electrode and a second working electrode, respectively. Therefore, the second and third electrodes are also referred to herein as working electrodes 104b and 104c and the first electrode as counter electrode 104a. Although, for the purpose of explanation only, electrochemical-based analytical test strip 100 is depicted as including a total of three electrodes, embodiments of electrochemical-based analytical test strips, including embodiments of the present invention, can include any suitable number of electrodes.

Patterned electrically conductive layer 104, including first electrode 104a, second electrode 104b and third electrode 104c, of electrochemical-based analytical test strip 100 can be formed of any suitable conductive material including, for example, electrically conducting carbon-based materials including carbon inks. It should be noted that patterned electrically conductive layers employed in electrochemical-based analytical test strips according to embodiments of the present invention can take any suitable shape and be formed of any suitable materials including, for example, metal materials and conductive carbon materials.

Referring in particular to FIGS. 1 and 2, the disposition of first electrode 104a, second electrode 104b and third electrode 104c and enzymatic reagent layer 108 are such that electrochemical-based analytical test strip 100 is configured for the electrochemical determination of an analyte (such as glucose) in a bodily fluid sample (such as a whole blood sample) that has filled sample-receiving chamber 118. The direction of such whole blood filling is depicted by the arrow labelled “S” in FIG. 2.

Enzymatic reagent layer 108 is disposed on at least a portion of patterned electrically conductor layer 104 (see FIGS. 1 and 2). Enzymatic reagent layer 108 includes at least one naphthoquinone-based mediator and FAD-GDH enzyme. The term “napthoquinone-based mediator” as used herein refers to a mediator of the form:

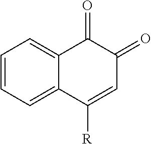

where R is any suitable organic substituent.

Particularly beneficial examples of naphthoquinone-based mediators suitable for use in embodiments of the present invention are 1,2-naphthalenedione-4-(3-mercapto-1-propane sulfonic acid) [also referred to herein as Compound A] and 1,2-naphthalenedione-4-(3-mercaptopropionic acid) [also referred to herein as Compound B]. However, once apprised of the present invention, one of skill in the art could employ routine experimentation to select suitable organic substituents (i.e., suitable “R” groups) to create other naphthoquinone-based mediators that have beneficial reaction kinetics, suitable aqueous solubility and that are not susceptible to interference effects. In this respect, a suitable R group may, for example, be sufficiently hydrophilic to impart a suitable aqueous solubility to the mediator.

Compound A has the molecular formula C₁₃H₁₂S₂O₅, a molecular weight of 312.36 and the following structure:

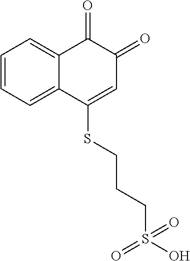 [SMILES: O=C1C=C(SCCCS(=O)(=O)O)c2ccccc2C1=O]

Compound A is an orange amorphous solid, soluble in base and neutral phosphate buffer at room temperature. Compound A has the following beneficial electrochemical characteristics:
E₀=0.086V vs Ag/AgCl (0.1M KCl)
k_{cat}=900 s⁻¹
   where E₀ is the formal redox potential vs Ag/AgCl (compare to 0.25V for ferricyanide) and k_{cat} is the rate constant for homogeneous electron transfer between the mediator (i.e., Compound A) and enzyme cofactor FAD. It is noted that E₀ for ferricyanide is 0.25V and k_{cat} for ferricyanide is 230 s⁻¹. Compound A has both an E₀ that is beneficially lower than that of ferricyanide (and thus less is less susceptible to interferents) and a k_{cat} that is beneficially higher than that of ferricyanide. Moreover, Compound A exhibited no reduction in glucose sensitivity after storage for 26 days at 40 degrees Celcius.

Compound A was synthesized as follows. 1,2-Naphthoquinone-4-sulfonic acid sodium salt (10 mmol) was added to distilled water (100 ml) and stirred until a clear solution developed. 3-Mercapto-1-propanesulfonic acid monosodium salt (10 mmol) was added at once creating a dark brown solution. The solution stood for 12 hours after which the clear brown solution was evaporated to dryness to yield a dark yellow solid. The dark yellow solid was washed with chloroform and dried under vacuum to yield 1,2-naphthalenedione-4-(3-mercapto-1-propane sulfonic acid) (1.48 g, 47%) as an orange solid. Mass spectra analysis confirmed the composition. However, once apprised of the present disclosure, one skilled in the art may develop other methods for synthesizing Compound A.

Compound B has the molecular formula C₁₃H₁₀S₂O₄, a molecular weight of 262.28 and the following structure:

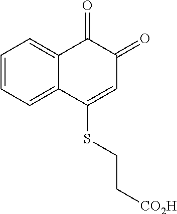 [SMILES: O=C(O)CCSC1=CC(=O)C(=O)c2ccccc21]

Compound B is an orange amorphous solid, soluble in base and neutral phosphate buffer at room temperature. Compound A has the following beneficial electrochemical characteristics:
E₀=0.016V vs Ag/AgCl (0.1M KCl)
k_{cat}=100 s⁻¹
   where E₀ is the formal redox potential vs Ag/AgCl (compare to 0.25V for ferricyanide) and k_{cat} is the rate constant for homogeneous electron transfer between the mediator (i.e., Compound A) and enzyme cofactor FAD. It is noted that E₀ for ferricyanide is 0.25V and k_{cat} for ferricyanide is 230 s⁻¹. Compound B has both an E₀ that is beneficially lower than that of ferricyanide (and thus less is less susceptible to interferents) and a k_{cat} that is lower than that of ferricyanide but still suitable. Moreover, Compound B exhibited no reduction in glucose sensitivity after storage for 26 days at 40 degrees Celcius.

Compound B was synthesized as follows. 1,2-Naphthoquinone (10 mmol) was added to methanol (50 ml) and stirred for 5 minutes in ice. 3-Mercaptopropionic acid (10 mmol) was added and within 5 minutes the solution went clear and was evaporated to dryness. The dark brown solid was purified in silica gel using ethyl acetate as eluent to yield 1,2-naphthalenedione-4-(3-mercaptopropionic acid) (1.19 g, 51%). Mass spectra analysis confirmed the composition. However, once apprised of the present disclosure, one skilled in the art may develop other methods for synthesizing Compound B.

An exemplary, but non-limiting, example of an enzymatic reagent according to the present invention that includes Compound B includes the following components:
10 ml of 0.1M aqueous phosphate solution buffered to pH 7, to which has been added:
   1% (w/v) Pluronic 103, a di-functional block copolymer surfactant terminating in primary hydroxyl groups
   2.5% (w/v) hydroxylethyl cellose, a thickening agent;
   40 mM Compound B as a final concentration
   0.05 g FAD-GDH
      This enzymatic reagent is also referred to herein as “Compound B enzymatic reagent.”

Another exemplary, but non-limiting example of an enzymatic reagent according to the present invention includes Compound A as follows:
100 mL of 0.01M aqueous phosphate solution buffered to pH 7 to which had been added:
   1% (w/v) silica
   1% (w/v) Pluronic 103
   5 drops of Dow Corning Antifoam 1500 (commercially available from VWR International, United Kingdom)
   5% hydroxyethyl cellulose.
Following orbital mixing, the following was added:
   175 mM final concentration of Compound A
   0.33 g FAD-GDH supplied by Amano Enzyme China Ltd
      This enzymatic reagent is also referred to herein as “Compound A enzymatic reagent.”

Referring in particular to FIGS. 3 and 4, analytical test strips employing Compound B enzymatic reagent were prepared with the enzymatic reagent applied during preparation using an ink-jet technique. Such ink-let techniques typically result in a final dry enzymatic layer thickness in the range of approximately 0.1 microns to 5 microns. The results of testing the resulting analytical test strips are depicted in FIGS. 3 and 4, which illustrate the beneficial linearity and lack of interference from uric acid of analytical test strips according to embodiments of the present invention.

The data of FIG. 3 was gathered using spiked whole venous blood from 3-representative donors with nominal hematocrit (i.e., in the range of 38% to 42%). The data of FIG. 4 compares the performance of the test strips against a commercially available electrochemical-based analytical test strip and was performed using single donor blood with a YSI plasma glucose concentration of 65 mg/dL. Uric acid was spiked into the blood to a final concentration of 22 mg·dL. Bias values to YSI plasma glucose levels were calculated and plotted as an increase in an associated test meter glucose signal relative to an un-spiked control. FIG. 4 illustrates the lack interference effects for analytical test strips that employ Compound B enzymatic reagent. Further studies not detailed here show similar lack of interference from acetaminophen, glutathione and ascorbic acid.

Referring in particular to FIG. 5, analytical test strips employing Compound A enzymatic reagent were prepared with the enzymatic reagent applied during preparation using a screen printing technique. Such screen printing techniques typically result in a final dry enzymatic layer thickness in the range of approximately 5 microns to 15 microns. The results of testing the resulting analytical test strips are depicted in FIG. 5, which illustrate the beneficial linearity of analytical test strips according to another embodiment of the present invention. The data of FIG. 5 was gathered using spiked whole venous blood from 3-representative donors with nominal hematocrit (i.e., in the range of 38% to 42%).

Once apprised of the present disclosure, one skilled in the art will recognize that a variety of suitable enzymatic reagents containing Compound A and/or Compound B and FAD-GDH can be formulated. Such suitable enzymatic reagents can include, for example, tri-sodium citrate, citric acid, polyvinyl alcohol, hydroxyl ethyl cellulose, antifoam, fumed silica (either with or without a hydrophobic surface modification), PVPVA, and water. Further details regarding reagent layers in general, and electrochemical-based analytical test strips in general, are in U.S. Pat. Nos. 6,241,862 and 6,733,655, the contents of which are hereby fully incorporated by reference.

Referring to FIGS. 1 and 2, patterned insulation layer 106 can be formed of any suitable electrically-insulating dielectric material including commercially available screen-printable dielectric inks.

Patterned spacer layer 110 can be formed, for example, from a screen-printable pressure sensitive adhesive commercially available from Apollo Adhesives, Tamworth, Staffordshire, UK. In the embodiment of FIG. and 2, patterned spacer layer 110 defines outer walls of the sample-receiving chamber 118. Patterned spacer layer 110 can have a thickness of, for example, approximately 110 microns, be electrically nonconductive, and be formed of a polyester material with top and bottom side acrylic-based pressure sensitive adhesive.

Top layer 112 can be, for example, a clear film with hydrophilic properties that promote wetting and filling of electrochemical-based analytical test strip 100 by a fluid sample (e.g., a whole blood sample). Such clear films are commercially available from, for example, 3M of Minneapolis, Minn. U.S.A. and Coveme (San Lazzaro di Savena, Italy). Top layer 112 can be, for example, a polyester film coated with a surfactant that provides a hydrophilic contact angle <10 degrees. Top layer 112 can also be a polypropylene film coated with a surfactant or other surface treatment. In such a circumstance, the surfactant coating serves as hydrophilic sub-layer 114. Top layer 112 can have a thickness, for example, of approximately 100 μm.

Electrochemical-based analytical test strip 100 can be manufactured, for example, by the sequential aligned formation of patterned electrically conductive layer 104, patterned insulating layer 106, enzymatic reagent layer 108, patterned spacer layer 110, and top layer 112. Any suitable techniques known to one skilled in the art can be used to accomplish such sequential aligned formation, including, for example, screen printing, ink-jet printing, photolithography, photogravure, chemical vapour deposition and tape lamination techniques. However, enzymatic reagents according to embodiments of the present invention are particularly beneficial in that they can be formulated as aqueous compositions suitable for relatively low-cost and otherwise conventional ink jet and screen printing techniques. Such enzymatic reagents can, for example, be employed to create enzymatic reagent layers with a linear response between electrochemically-generated current and glucose concentration in a whole blood sample up to at least a glucose concentration of 700 mg/dL.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and compositions of matter within the scope of these claims and their equivalents be covered thereby.

What is claimed is:

1. An electrochemical-based analytical test strip for the determination of an analyte in a bodily fluid sample, the electrochemical-based analytical test strip comprising:
an electrically insulating base layer;
an electrically conductive layer disposed on the electrically insulating base layer and including at least one electrode;
an enzymatic reagent layer disposed on the at least one electrode;
a patterned spacer layer; and
a top layer.
wherein the enzymatic reagent layer includes:
at least one of the naphthoquinone-based mediator of the form: 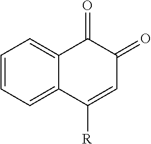
where R=an organic substituent; and
and
FAD-GDH enzyme.

2. The electrochemical-based analytical test strip of claim 1 wherein the at least one naphthoquinone-based mediator is at least one of the mediators:
1,2-naphthalenedione-4-(3-mercapto-1-propane sulfonic acid) and
1,2-naphthalenedione-4-(3-mercaptopropionic acid);

3. The electrochemical-based analytical test strip of claim 2 wherein the at least one of 1,2-naphthalenedione-4-(3-mercapto-1-propane sulfonic acid) and 1,2-naphthalenedione-4-(3-mercaptopropionic acid) is 1,2-naphthalenedione-4-(3-mercapto-1-propane sulfonic acid).

4. The electrochemical-based analytical test strip of claim 3 wherein the enzymatic reagent layer further includes:
a dried aqueous phosphate solution buffered to Ph 7;
a surfactant; and
a thickening agent.

5. The electrochemical-based analytical test strip of claim 3 wherein 1,2-naphthalenedione-4-(3-mercapto-1-propane sulfonic acid) is present at a concentration of approximately 0.175 mM during application to the electrochemical-based analytical test strip.

6. The electrochemical-based analytical test strip of claim 3 wherein the enzymatic reagent layer is applied using a screen printing technique and has a thickness in the range of approximately 5 microns to 15 microns.

7. The electrochemical-based analytical test strip of claim 2 wherein the at least one of 1,2-naphthalenedione-4-(3-mercapto-1-propane sulfonic acid) and 1,2-naphthalenedione-4-(3-mercaptopropionic acid) is 1,2-naphthalenedione-4-(3-mercaptopropionic acid).

8. The electrochemical-based analytical test strip of claim 7 wherein the enzymatic reagent layer further includes:
a surfactant; and
a thickening agent.

9. The electrochemical-based analytical test strip of claim 6 wherein 1,2-naphthalenedione-4-(3-mercaptopropionic acid) is present at a concentration in the range of approximately 40 mMmM during application to the electrochemical-based test strip.

10. The electrochemical-based analytical test strip of claim 7 wherein the enzymatic reagent layer is applied using an ink-jet technique and has a thickness in the range pf 0.1 microns to 5.0 microns.

11. The electrochemical-based analytical test strip of claim 1 wherein the bodily fluid sample is a whole blood sample and the analyte is glucose.

12. An enzymatic reagent for use in electrochemical-based analytical test strips, the enzymatic reagent comprising:
at least one mediator of the form:
1,2-naphthalenedione-4-(3-mercapto-1-propane sulfonic acid); and
1,2-naphthalenedione-4-(3-mercaptopropionic acid); and FAD-GDH enzyme.

13. The enzymatic reagent of claim 12, wherein the at least one mediator is at least one of:
1,2-naphthalenedione-4-(3-mercapto-1-propane sulfonic acid); and
1,2-naphthalenedione-4-(3-mercaptopropionic acid)

14. The enzymatic reagent of claim 13 wherein the at least one of 1,2-naphthalenedione-4-(3-mercapto-1-propane sulfonic acid) and 1,2-naphthalenedione-4-(3-mercaptopropionic acid) is 1,2-naphthalenedione-4-(3-mercapto-1-propane sulfonic acid).

15. The enzymatic reagent of claim 12 further including:
an aqueous phosphate solution buffered to Ph 7;
a surfactant; and
a thickening agent.

16. The enzymatic reagent of claim 13 wherein 1,2-naphthalenedione-4-(3-mercapto-1-propane sulfonic acid) is present at a concentration of approximately 175 mM.

17. The enzymatic reagent of claim 15 further including an anti-foaming agent.

18. The enzymatic reagent of claim 13 wherein the at least one of 1,2-naphthalenedione-4-(3-mercapto-1-propane sulfonic acid) and 1,2-naphthalenedione-4-(3-mercaptopropionic acid) is 1,2-naphthalenedione-4-(3-mercaptopropionic acid).

19. The enzymatic reagent of claim 18 further including
an aqueous phosphate solution buffered to pH 7;
a surfactant; and
a thickening agent.

20. The enzymatic reagent of claim 18 wherein 1,2-naphthalenedione-4-(3-mercaptopropionic acid) is present at a concentration of approximately 40 mM.
